(19) United States
(12) Patent Application Publication
(10) Pub. No.: US 2016/0022131 A1
(43) Pub. Date: Jan. 28, 2016

(54) WATERPROOF LARYNGOSCOPE AND WATERPROOF STRUCTURE

(71) Applicants: IEI Integration Corp., New Taipei 221 (TW); ARMORLINK SH CORP., Shanghai (CN)

(72) Inventors: KAI-CHENG CHAN, NEW TAIPEI CITY 221 (TW); CHIN-CHIA CHANG, NEW TAIPEI CITY 221 (TW)

(21) Appl. No.: 14/557,643

(22) Filed: Dec. 2, 2014

(30) Foreign Application Priority Data
Jul. 22, 2014 (CN) 201410350452.2

(51) Int. Cl. A61B 1/267; A61B 1/00; A61B 1/04

(52) U.S. Cl. CPC A61B 1/267; A61B 1/04; A61B 1/00142

(57) ABSTRACT

A waterproof laryngoscope and a waterproof structure are revealed. The waterproof laryngoscope includes a laryngoscope body and a waterproof sleeve. The laryngoscope body consists of a handle and a camera tube. The handle has a fixing portion disposed with at least one stopper. The camera tube is fixed in the handle and extended out of the fixing portion. The waterproof sleeve is fitted over the camera tube and fixed on the handle. The waterproof sleeve includes a connection portion that is sleeved over the camera tube and fixed on the fixing portion. The connection portion is arranged with at least one fixing groove. The stopper is slid in the fixing groove and locked on a rear end of the fixing groove. Thereby not only patient's body fluid will not flow into the laryngoscope, patients are also protected from being affected by toxic chemicals and cross infection.

### BACKGROUND OF THE INVENTION

1. Fields of the Invention

The present invention relates to a laryngoscope, especially to a waterproof laryngoscope and a waterproof structure.

2. Descriptions of Related Art

Generally, many patients in clinic including patients with general anesthesia, patients in ICU (intensive care unit), emergency room, and other departments need tracheal intubation for respiration control. Most of tracheal intubation is performed by using a laryngoscope under anesthesia. Thus the laryngoscope requires washing and disinfection quite often for preventing cross infection among the patients.

According to recommendations of American Society of Anesthesiologists, the laryngoscope should be soaked in glutaraldehyde after being washed and cleaned thoroughly. Ethylene oxide is used by certain domestic medical institutions for disinfection. In some hospitals, the laryngoscope is just washed and cleaned. Both glutaraldehyde and ethylene oxide are toxic chemicals and should be used carefully. These chemicals also result in erosion of the laryngoscope and shorter life time of the laryngoscope. Moreover, the laryngoscope has been identified as a potential source of cross infection. Thus it should be washed, cleaned, followed by high-level disinfection. Only washing and alcohol disinfection procedure may increase the chance of cross infection.

The laryngoscope used now is also disposed with camera lens and micro-display. The camera lens is used to capture images and transmit the images to the display. Thus users can perform the operation more conveniently. Generally, the camera lens is arranged at the front end of the laryngoscope while the display is set on the side of a handle of the laryngoscope for users to monitor the images conveniently. There is a certain distance between the camera lens and the display so that the camera lens and the display are electrically connected to each other by transmission lines for image transmission. A camera tube is used to mount the transmission lines therein and connect the camera lens to the display electrically.

However, during the operations of the patients, the patient's body fluid is more or less infiltrated into the laryngoscope, maybe further into the camera tube. After a long run, the transmission lines and the camera lens get damaged.

Thus there is room for improvement and a need to provide an easy and effective design of the laryngoscope that not only prevents the laryngoscope from being damaged but also protects the patients without being affected by toxic chemical substances and cross infection.

### SUMMARY OF THE INVENTION

Therefore it is a primary object of the present invention to provide a waterproof laryngoscope and a waterproof structure that not only prevents patient's body fluid flowing into the laryngoscope for longer life time and better working condition of the laryngoscope but also protects patients from being affected by toxic chemical substances and cross infection. The waterproof structure includes a connection portion of a waterproof sleeve fitted over a camera tube of the laryngoscope and fixed on a fixing portion of a handle of the laryngoscope. A fixing groove is set on the connection portion for allowing stoppers slid therein and locked at a rear end thereof.

It is another object of the present invention to provide a waterproof laryngoscope and a waterproof structure in which water resistance of a waterproof sleeve is improved by a fixing groove arranged at a sidewall of a connection portion of the waterproof sleeve, without penetrating the sidewall of the connection portion.

It is a further object of the present invention to provide a waterproof laryngoscope and a waterproof structure with better water resistance by using a washer clipped between a fixing portion and a connection portion of the laryngoscope.

In order to achieve the above objects, a waterproof laryngoscope of the present invention is formed by a laryngoscope body and a waterproof sleeve. The laryngoscope body includes a handle and a camera tube. The handle has a fixing portion arranged with at least one stopper. The camera tube is fixed in the handle and is extended out of the fixing portion. The waterproof sleeve is fitted over the camera tube and fixed on the handle. The waterproof sleeve includes a connection portion that is sleeved over the camera tube and fixed on the fixing portion. The connection portion is arranged with at least one fixing groove. The stopper is slid in the fixing groove and locked on a rear end of the fixing groove. By a washer set between the fixing portion of the handle and the connection portion of the waterproof sleeve, and the stopper of the fixing portion locked on the fixing groove of the connection portion after rotation of the connection portion that presses the washer, the patient's body fluid will not flow into the laryngoscope. Thus the laryngoscope can be used under good condition for a long period of time. Moreover, the patient is also protected by the design, without being hurt by toxic chemical substances and cross infection.

Furthermore, the washer of the waterproof laryngoscope is disposed with a fixing hole. The camera tube is inserted through the washer so that the washer is clipped between the fixing portion and the connection portion. Thus the water resistance of the laryngoscope is improved.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a perspective view of an embodiment according to the present invention;

FIG. 2 is an explosive view of an embodiment according to the present invention;

FIG. 3 is a bottom view of a handle of an embodiment according to the present invention;

FIG. 4 is a rear view of a waterproof sleeve of an embodiment according to the present invention;

FIG. 5 is a schematic drawing showing a fixing groove of an embodiment according to the present invention;

FIG. 6 is a right side view of a waterproof sleeve of an embodiment according to the present invention;

FIG. 7 to FIG. 17 are schematic drawings showing the assembly of an embodiment according to the present invention;

FIG. 18 is a rear view of a waterproof sleeve of another embodiment according to the present invention;

FIG. 19 to FIG. 24 are schematic drawings showing the assembly of another embodiment according to the present invention;

FIG. 25 is a schematic drawing showing a fixing groove of another embodiment according to the present invention;

FIG. 26 is a schematic drawing showing a fixing groove of a further embodiment according to the present invention;

FIG. 27 is a schematic drawing showing a fixing groove of a further embodiment according to the present invention;

FIG. 28 is a schematic drawing showing a fixing groove of a further embodiment according to the present invention;

FIG. 29 is a schematic drawing showing a fixing groove of a further embodiment according to the present invention;

FIG. 30 is a schematic drawing showing a fixing groove of a further embodiment according to the present invention;

FIG. 31 is a schematic drawing showing a fixing groove of a further embodiment according to the present invention;

FIG. 32 is a schematic drawing showing a fixing groove of a further embodiment according to the present invention;

FIG. 33 is a schematic drawing showing a fixing groove of a further embodiment according to the present invention;

FIG. 34 is a bottom view of a handle of another embodiment according to the present invention;

FIG. 35 is a rear view of a waterproof sleeve of a further embodiment according to the present invention;

FIG. 36 is a schematic drawing showing an embodiment of the assembly of the handle in FIG. 34 with the waterproof sleeve in FIG. 35 according to the present invention;

FIG. 37 is a schematic drawing showing another embodiment of the assembly of the handle in FIG. 34 with the waterproof sleeve in FIG. 35 according to the present invention.

### DETAILED DESCRIPTION OF THE PREFERRED EMBODIMENT

Please refer to the following embodiment for more details of features and functions of the present invention.

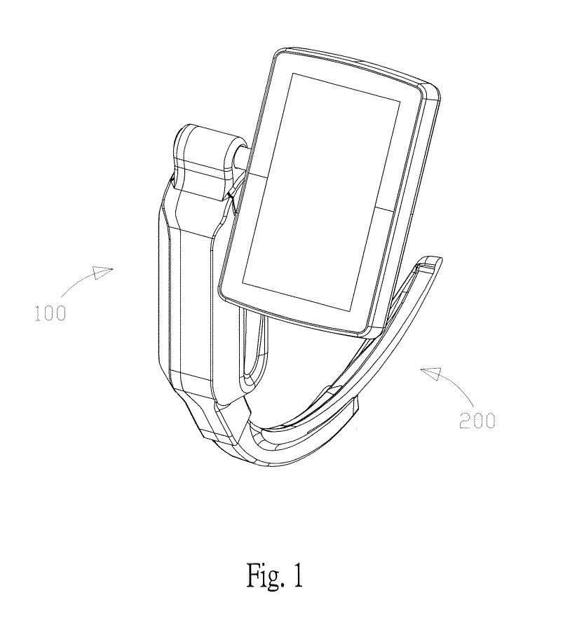

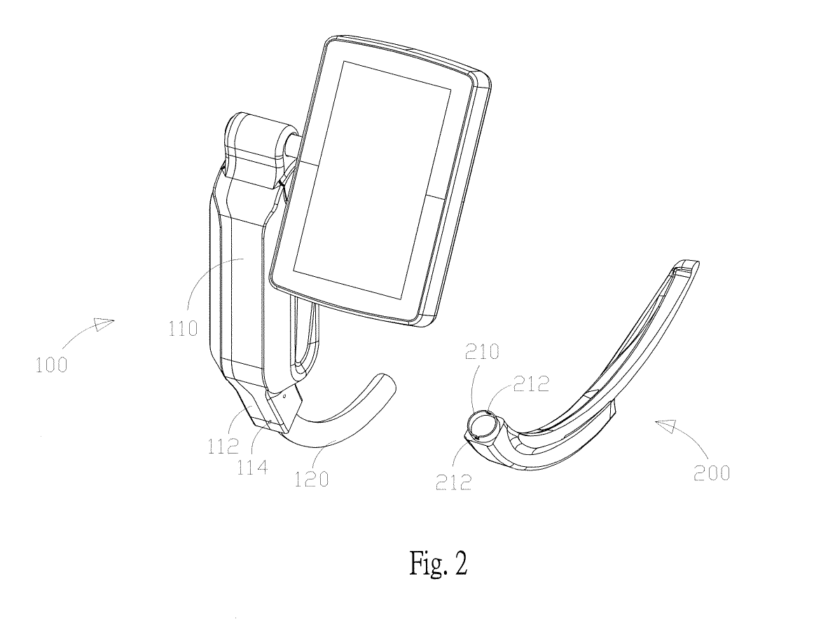

Refer from FIG. 1 to FIG. 4, a waterproof laryngoscope of the present invention includes a laryngoscope body 100 and a waterproof sleeve 200. The laryngoscope body 100 consists of a handle 110 and a camera tube 120. The handle 110 has a fixing portion 112 disposed with at least one stopper 114. In this embodiment, the fixing portion 112 is located under the handle 110 and is arranged with two stoppers 114. The number of the stoppers 114 on the fixing portion 112 is not limited. The two stoppers 114 are disposed on the two opposite sites respectively. The camera tube 120 is fixed in the handle 110 and is extended out of the fixing portion 112.

The waterproof sleeve 200 is fitted over the camera tube 120 and is fixed on the handle 110. The waterproof sleeve 200 has a connection portion 210 that is fitted over the camera tube 120 and is fixed on the fixing portion 112. The connection portion 210 is arranged with at least one fixing groove 212. The stopper 114 is slid in the fixing groove 212 and locked on a rear end of the fixing groove 212. The waterproof sleeve 200 is made from flexible material such as polyethylene (PE), polypropylene (PP), etc. By the connection portion 210 of the waterproof sleeve 200 fitted over the camera tube 120 and fixed on the fixing portion 112, and the stopper 114 slid in the fixing groove 212 of the connection portion 210 and locked on the rear end of the fixing groove 212, the patient's body fluid will not flow into the laryngoscope. Thus the laryngoscope is protected and is able to be used under good condition for a long period of time. Moreover, the patient is also protected, without being hurt by toxic chemical substances and cross infection.

The waterproof structure of the present invention includes the handle 110 and the waterproof sleeve 200. The handle 110 has the fixing portion 112 with at least one stopper 114. The waterproof sleeve 200 is fixed on the handle 110 and having a connection portion 210. The connection portion 210 is fixed on the fixing portion 112. The connection portion 210 is set with the fixing groove 212 while the stopper 114 is slid in the fixing groove 212 and locked on the rear end of the fixing groove 212. This is the waterproof structure. The waterproof structure of the present invention is not limited to be used on the laryngoscope. It can also be applied to other objects. Thus various techniques related to modifications and applications of the waterproof structure of the present invention are within the scope of the present invention.

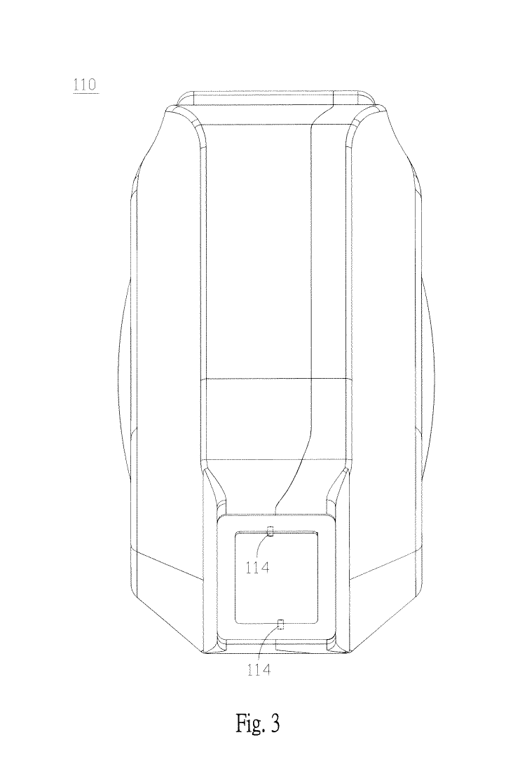

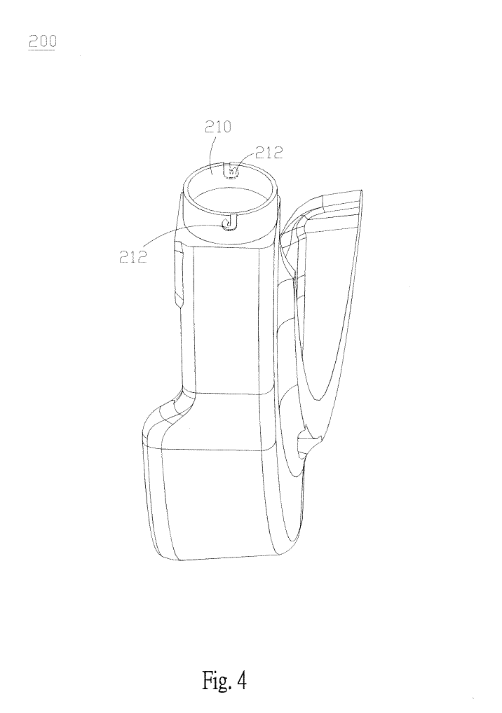

As shown in FIG. 3 and FIG. 4, the fixing portion 112 of the handle 110 is a first receiving slot while the connection portion 210 of the waterproof sleeve 200 is a first hollow cylinder. The first hollow cylinder is mounted in the first receiving slot. The stoppers 114 are disposed on an inner sidewall of the first receiving slot while the fixing grooves 212 are formed on an outer sidewall of the first hollow cylinder. The positions of the stoppers 114 on the first receiving slot are corresponding to the positions of the fixing grooves 212 on the first hollow cylinder so that the stoppers 114 are slid in the fixing grooves 212 to and locked on the rear end of the fixing grooves 212 respectively.

In this embodiment, the fixing grooves 212 are formed on an outer sidewall of the first hollow cylinder without penetrating the outer sidewall of the first hollow cylinder. Thus the outer sidewall prevents the fluid from infiltrating into the laryngoscope body 100 through the fixing grooves 212. The water resistance of the waterproof sleeve 200 is enhanced by the disposition of the fixing grooves 212 on the sidewall of the connection portion 210 of the waterproof sleeve 200 without penetrating the sidewall of the connection portion 210.

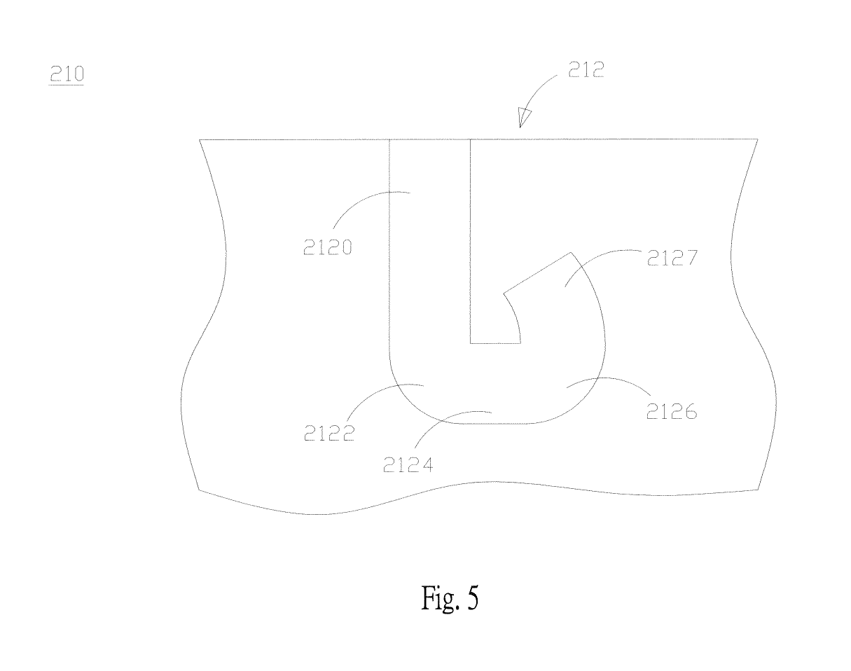

Refer to FIG. 5, a schematic drawing showing the fixing groove 212 of an embodiment of the present invention is revealed. As shown in the figure, the fixing groove 212 consists of a guiding part 2120, a first turning part 2122, a sliding part 2124, a second turning part 2126 and a holding and locking part 2127. The guiding part 2120 is disposed on the outer sidewall of the connection portion 210 and crossed over a top edge of the outer sidewall of the connection portion 210. One end of the first turning part 2122 is connected to the guiding part 2120 while the other end thereof is connected to the sliding part 2124. An angle between the sliding part 2124 and the guiding part 2120 is no less than 90 degrees. The second turning part 2126 is connected to the sliding part 2124. The turning direction of the second turning part 2126 is the same as that of the first turning part 2122. The holding and locking part 2127 is connected to the second turning part 2126. Thereby the stopper 114 of the fixing portion 112 is set above the guiding part 2120, passed through the first turning part 2122, the sliding part 2124, and the second turning part 2126 to be locked on the holding and locking part 2127. How the waterproof sleeve 200 is fitted over the laryngoscope body 100 is described in details in the following.

Moreover, the first turning part 2122 as well as the second turning part 2126 of the fixing groove 212 in this embodiment is a fillet. Thus the user slides the stopper 114 inside the fixing groove 212 and allows the stopper 114 being locked on the rear end of the fixing groove 212 easily with a lighter force.

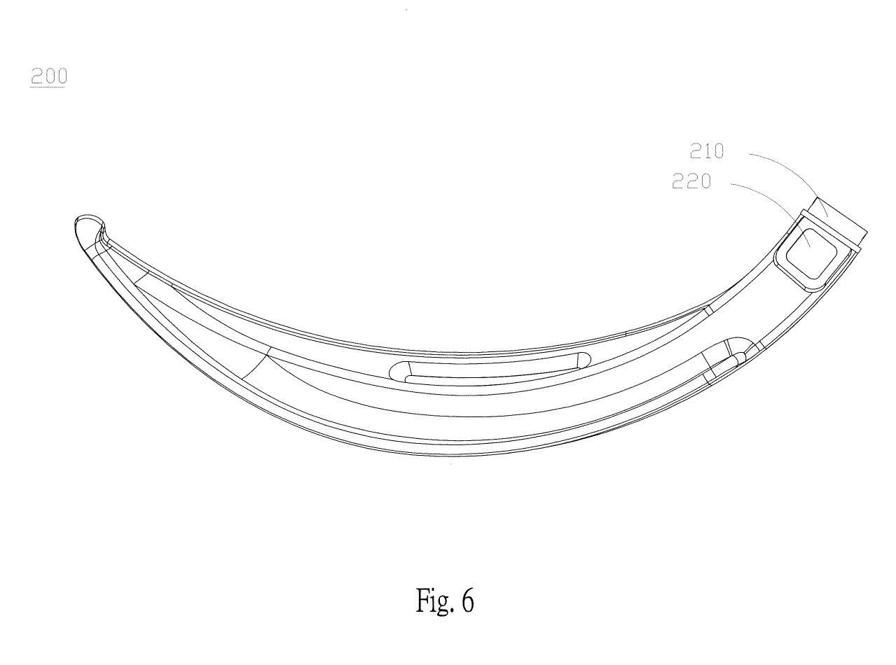

Refer to FIG. 6, a right side view of a waterproof sleeve 200 of an embodiment according to the present invention is revealed. The waterproof sleeve 200 includes a non-slip portion 220 disposed on the right side thereof, near the connection portion 210. Thus the user can fit the waterproof sleeve 200 over the fixing portion 112 of the laryngoscope body 100 conveniently.

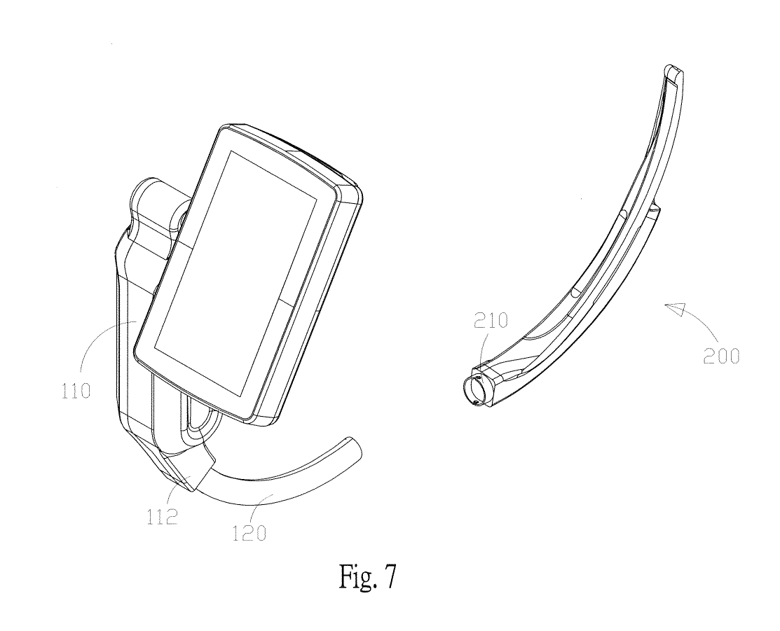

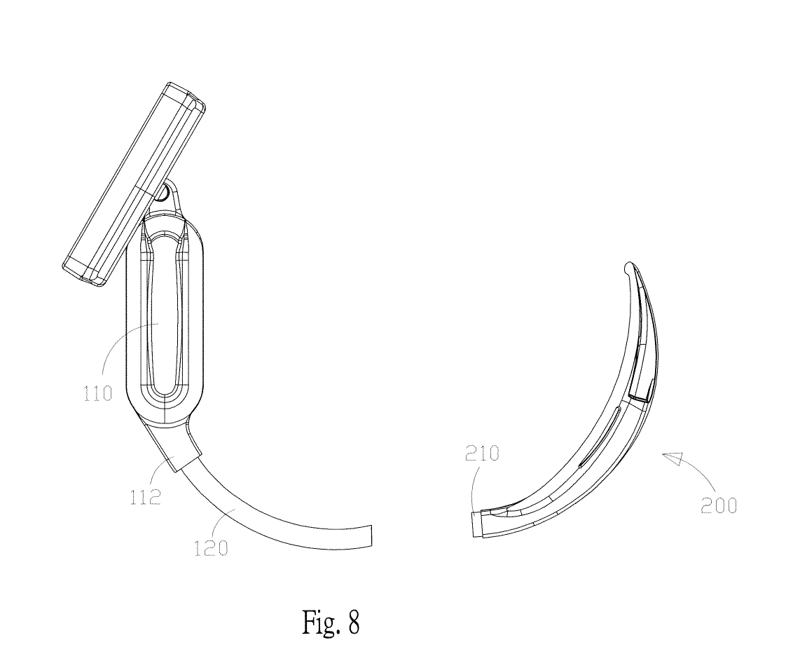

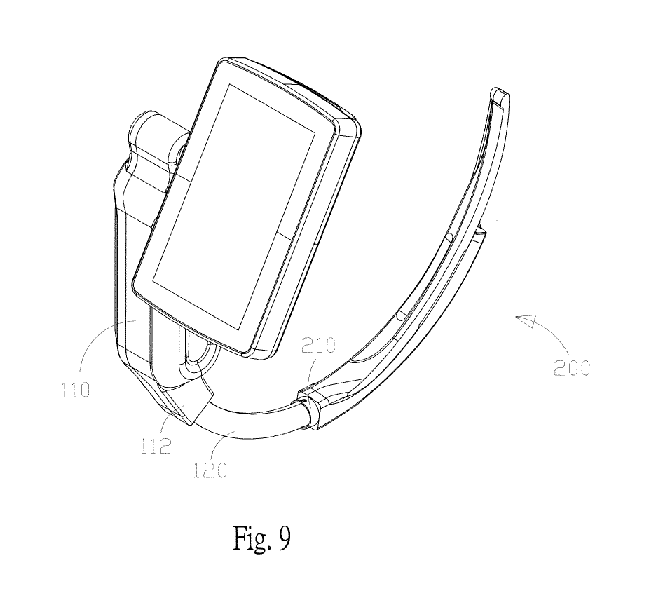

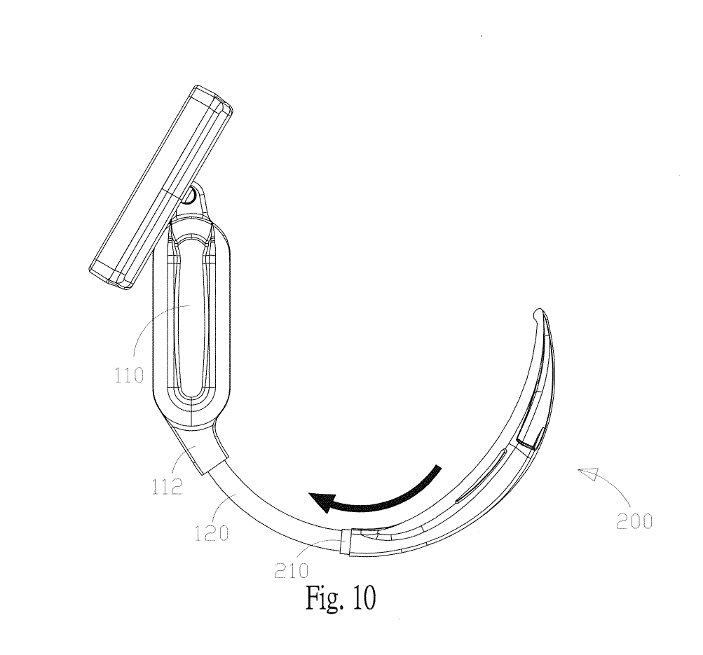

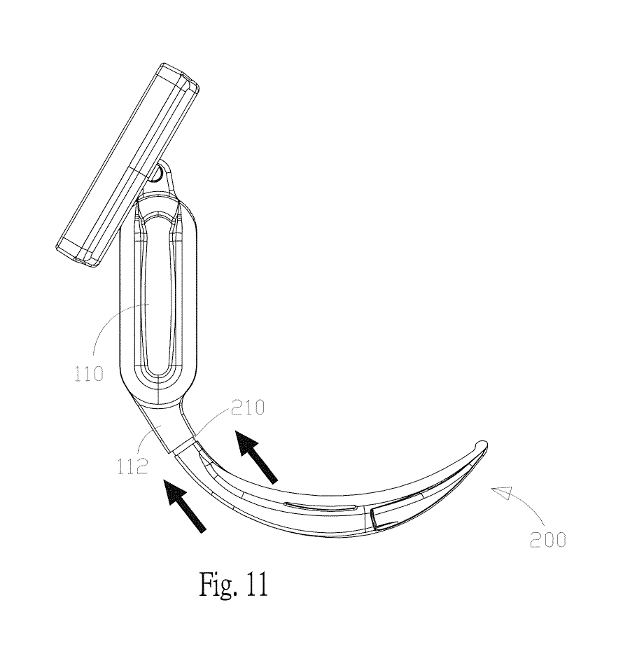

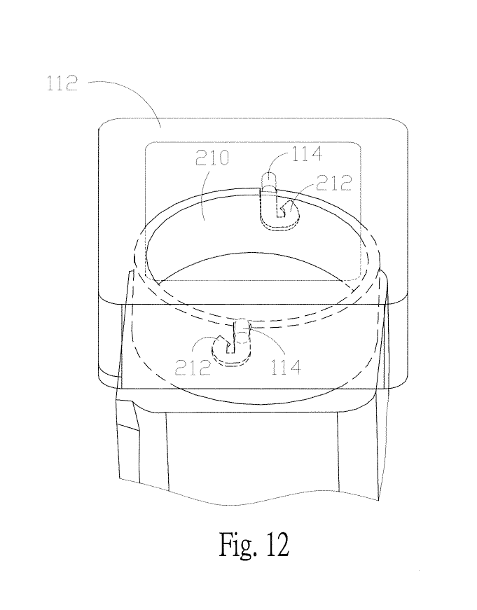

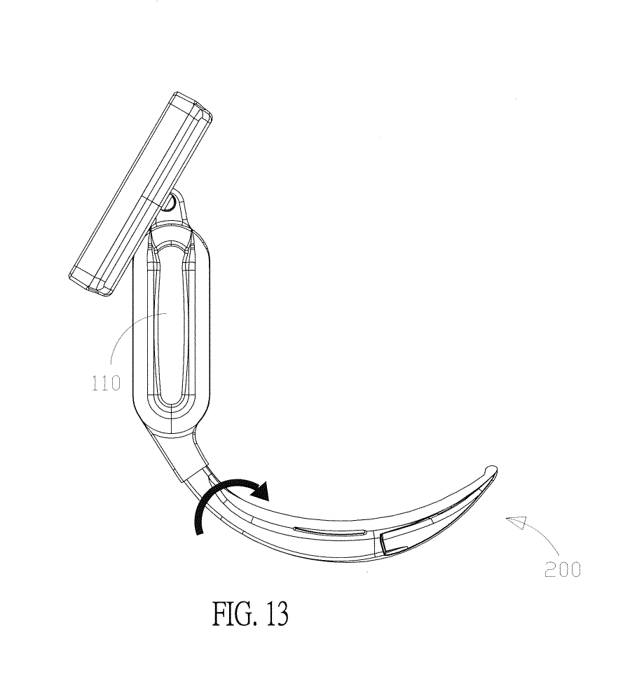

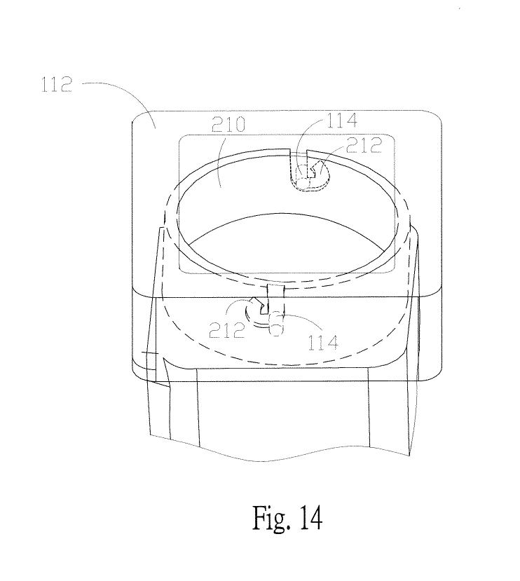

Refer from FIG. 7 to FIG. 17, schematic drawings showing the assembly of an embodiment of a laryngoscope are revealed. In this embodiment, the connection portion 210 of the waterproof sleeve 200 is fitted over the camera tube 120 and then is fixed on the fixing portion 112 of the handle 110. As shown in FIG. 7 and FIG. 8, the connection portion 210 of the waterproof sleeve 200 is aligned with the camera tube 120 so as to mount the camera tube 120 in the waterproof sleeve 200. Then refer to FIG. 9 and FIG. 10, a part of the camera tube 120 has already been sleeved in the waterproof sleeve 200 and the rest part is continuingly fitted into the waterproof sleeve 200 until the waterproof sleeve 200 is in contact with the fixing portion 112 of the handle 110. Next refer to FIG. 11 and FIG. 12, the waterproof sleeve 200 is pressed toward the fixing portion 112 of the handle 110 so as to allow each stopper 114 of the fixing portion 112 entering the guiding part 2120 of the corresponding fixing groove 212 of the connection portion 210 and moving along the guiding part 2120 to reach the first turning part 2122. As shown in FIG. 13 and FIG. 14, when the waterproof sleeve 200 is pressed to allow the stoppers 114 reaching the first turning part 2122, rotate the waterproof sleeve 200 clockwise or counterclockwise to make the stopper 114 enter and slide in the sliding part 2124 correspondingly. In this embodiment, the waterproof sleeve 200 is rotated counterclockwise to allow the stopper 114 slid in the sliding part 2124.

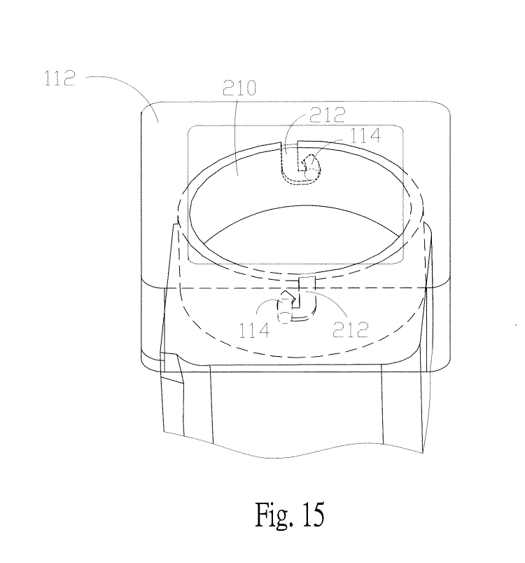

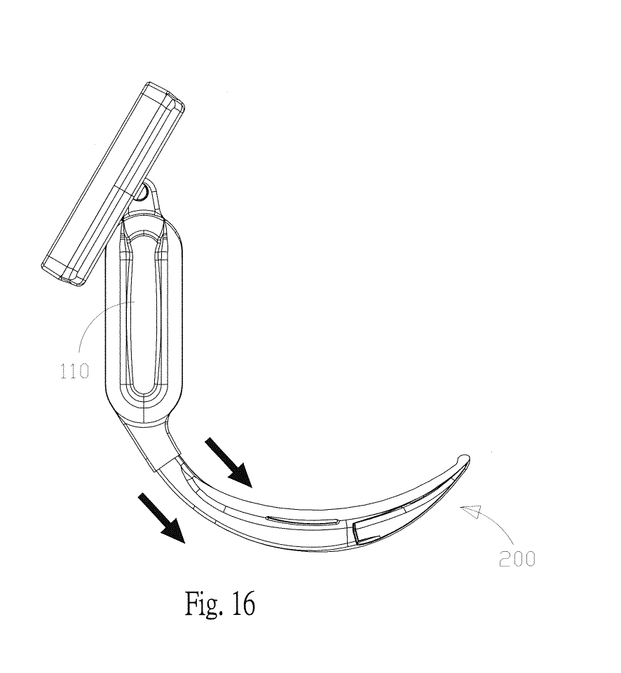

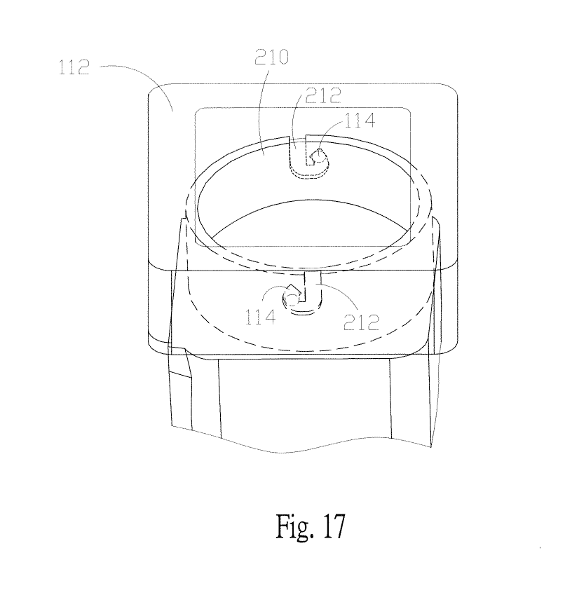

Then refer to FIG. 15, when the waterproof sleeve 200 is continuingly rotated counterclockwise until it is against something. This means the stopper 114 has reached the second turning part 2126. As shown in FIG. 16 and FIG. 17, the waterproof sleeve 200 is pulled backward along the direction of the opening of the fixing portion 112 of the handle 110 when the stopper 114 is against the second turning part 2126. Thus the stopper 114 is moved along the second turning part 2126 to lean against the holding and locking part 2127. Due to flexible material of the waterproof sleeve 200, the position of the stopper 114 (the distance between the stopper 114 and the opening of the fixing portion 112) and the length difference between the stopper 114 and the guiding part 2120 of the fixing groove 212, the stopper 114 is locked and fixed on the holding and locking part 2127 while being turned into the holding and locking part 2127. Thus the connection portion 210 of the waterproof sleeve 200 is tightly attached to the fixing portion 112 of the handle 110. Therefore the patient's body fluid will not flow into the laryngoscope. The laryngoscope can work properly for a long period of time. The patient is also protected, without being affected by toxic chemical substances and cross infection.

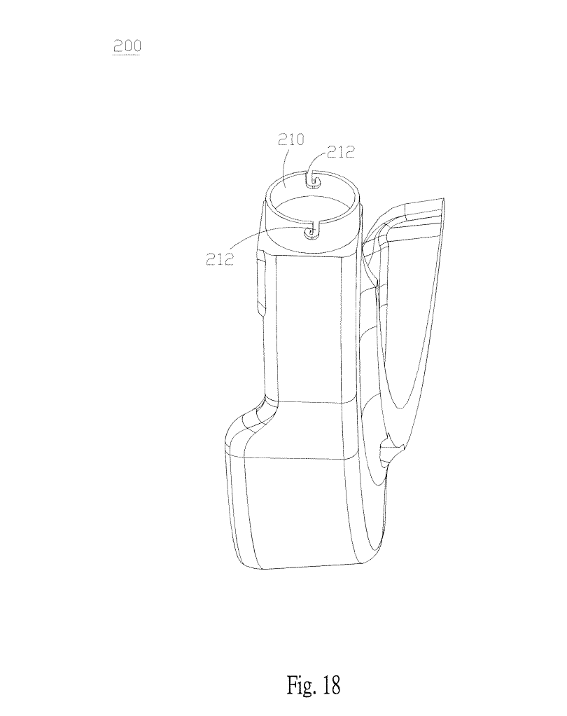

Refer to FIG. 18, a rear view of a waterproof sleeve of another embodiment is revealed. The difference between the waterproof sleeve 200 in this embodiment and the embodiment shown in FIG. 4 is in that the fixing groove 212 of the connection portion 210 of the waterproof sleeve 200 is arranged at an outer sidewall of the connection portion 210. Moreover, the fixing groove 212 is penetrating the outer sidewall of the connection portion 210. No matter the fixing groove 212 is penetrating the sidewall of the connection portion 210, it's within the scope of the present invention.

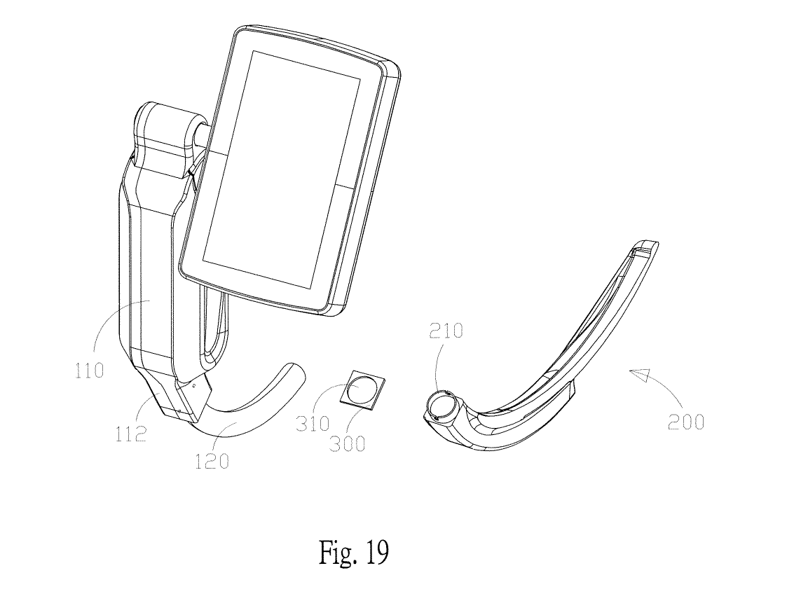

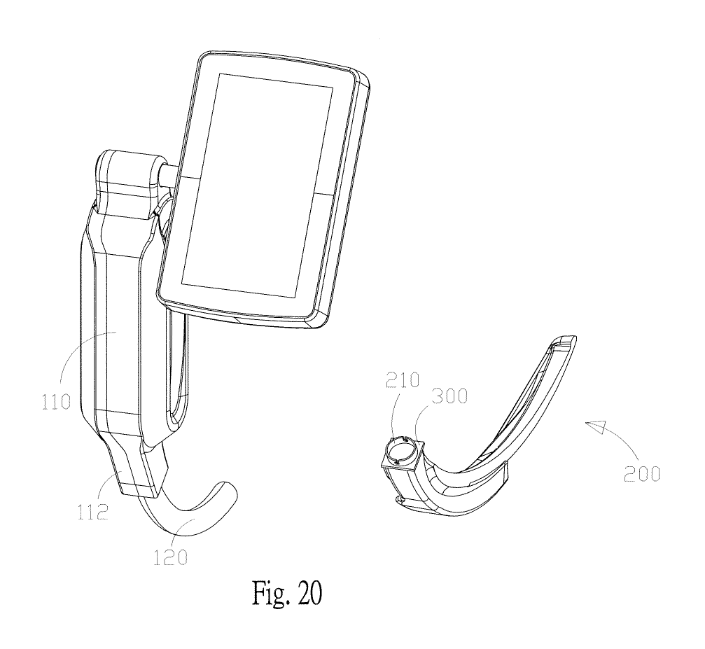

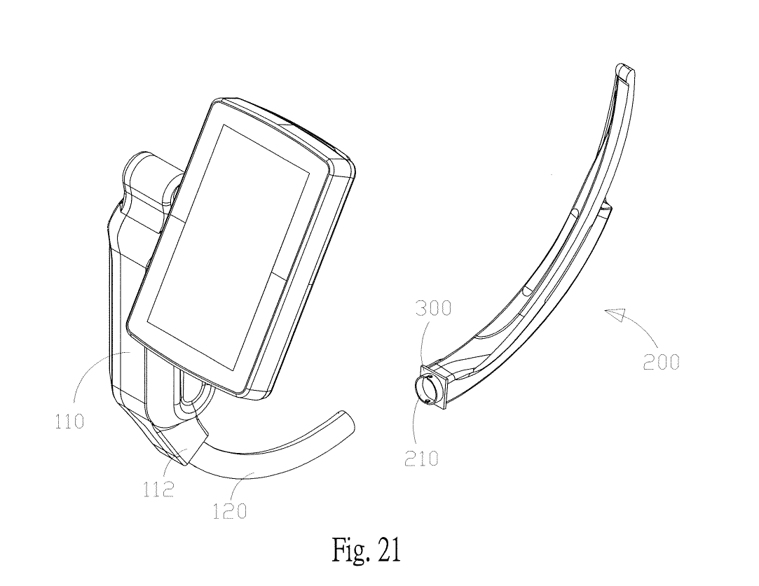

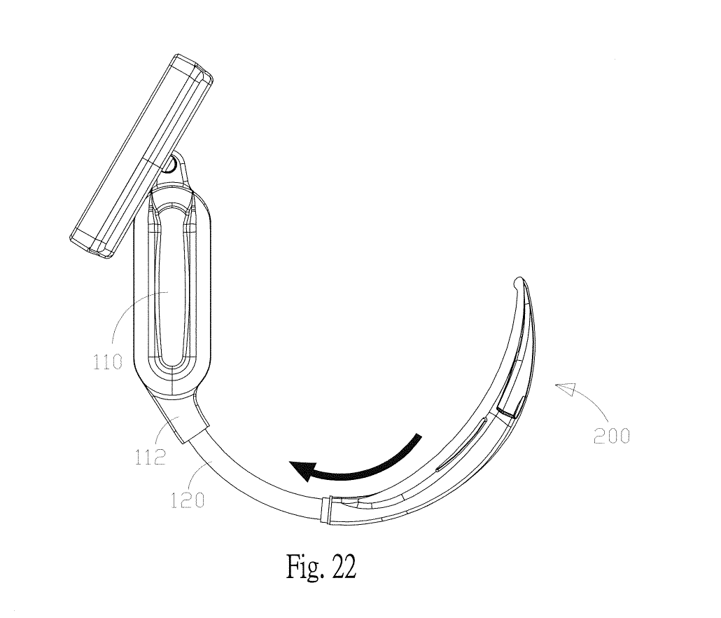

Refer from FIG. 19 to FIG. 24, schematic drawings showing the assembly of another embodiment according to the present invention are disclosed. The difference between this embodiment and the embodiment in FIG. 7 to FIG. 17 is in that the laryngoscope of this embodiment further includes a washer 300 with a fixing hole 310. The camera tube 120 is inserted through the washer 300. The washer 300 is clipped between the fixing portion 112 of the handle 110 and the connection portion 210 of the waterproof sleeve 200. By the washer 300 between the fixing portion 112 and the connection portion 210, the water resistance of the laryngoscope is enhanced. As shown from FIG. 19 to FIG. 21, first a laryngoscope body 100, a washer 300, and a waterproof sleeve 200 are arranged from the left side to the right side in turn for assembly of the laryngoscope body 100. A washer 300 is set on the waterproof sleeve 200 by inserting the connection portion 210 through the washer 300. Then the connection portion 210 of the waterproof sleeve 200 is aligned with the camera tube 120 and the waterproof sleeve 200 is sleeved over the camera tube 120 until the waterproof sleeve 200 is in contact with the fixing portion 112 of the handle 110.

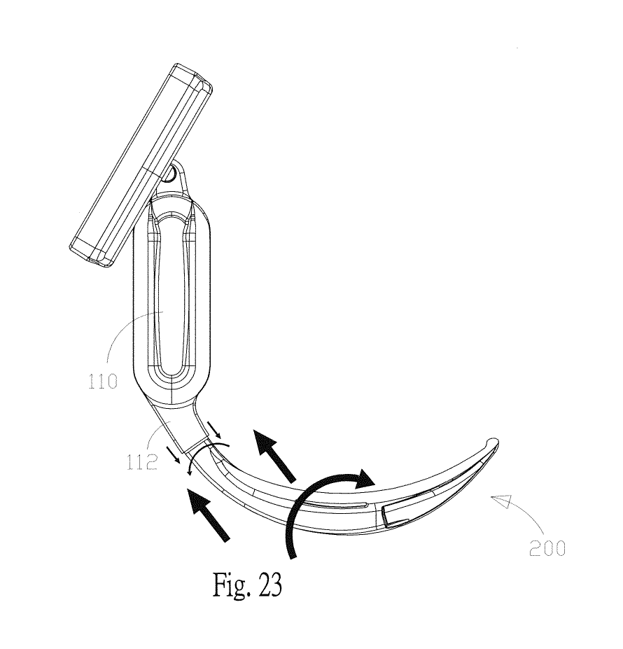

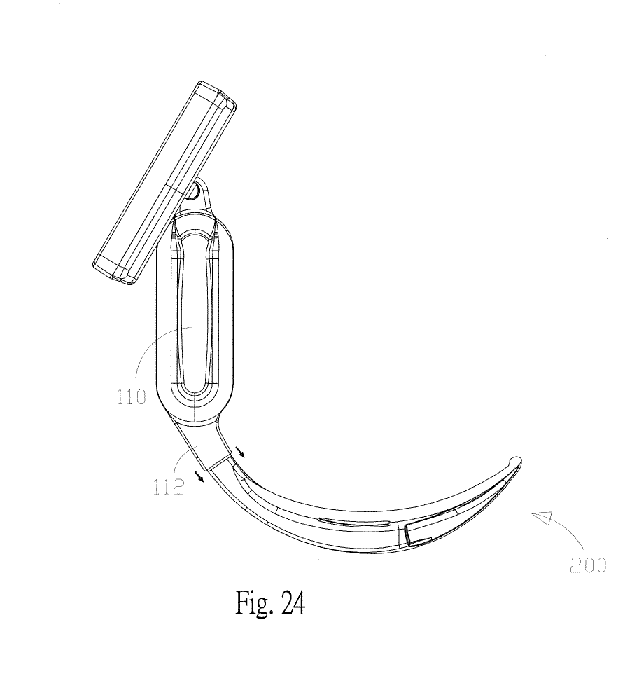

As shown in FIG. 23, the waterproof sleeve 200 is pressed toward the fixing portion 122 and a force is applied to the waterproof sleeve 200 clockwise at the same time to rotate the waterproof sleeve 200 clockwise (the large arrow in the figure represents the direction of the force). Thus the stopper 114 is against the holding and locking part 2127. Due to the washer 300 located between the fixing portion 112 and the connection portion 210, a reaction force is exerted on the washer 300 (as the small arrows indicate in the figure) by the waterproof sleeve 200 when the waterproof sleeve 200 is pressed the fixing portion 112 and a force is applied clockwise. Refer to FIG. 24, the washer 300 exerts an action on the waterproof sleeve 200 due to the washer 300 being pressed by the waterproof sleeve 200 and located between the fixing portion 112 and the connection portion 210. This action force allows the stopper 114 being fixed on the holding and locking part 2127 of the fixing groove 212. The design prevents the patient's body fluid from flowing into the laryngoscope for protection of the laryngoscope. The patient is also protected by the design, without being affected by toxic chemical substances and cross section.

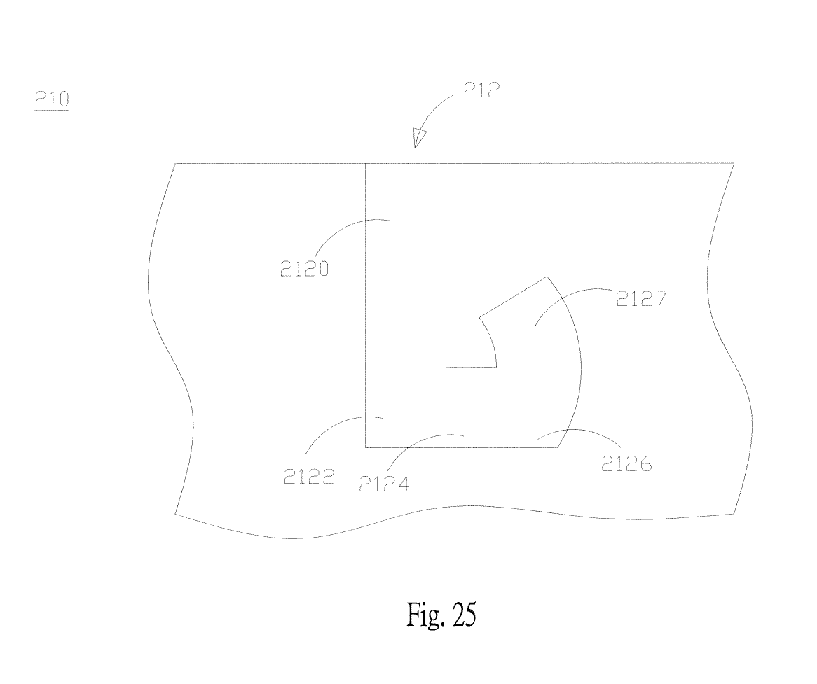

Refer to FIG. 25, a schematic drawing showing a fixing groove of another embodiment according to the present invention is revealed. The difference between this embodiment and the embodiment in FIG. 5 is in that the first turning part 2122 as well as the second turning part 2126 of this embodiment is a right angle.

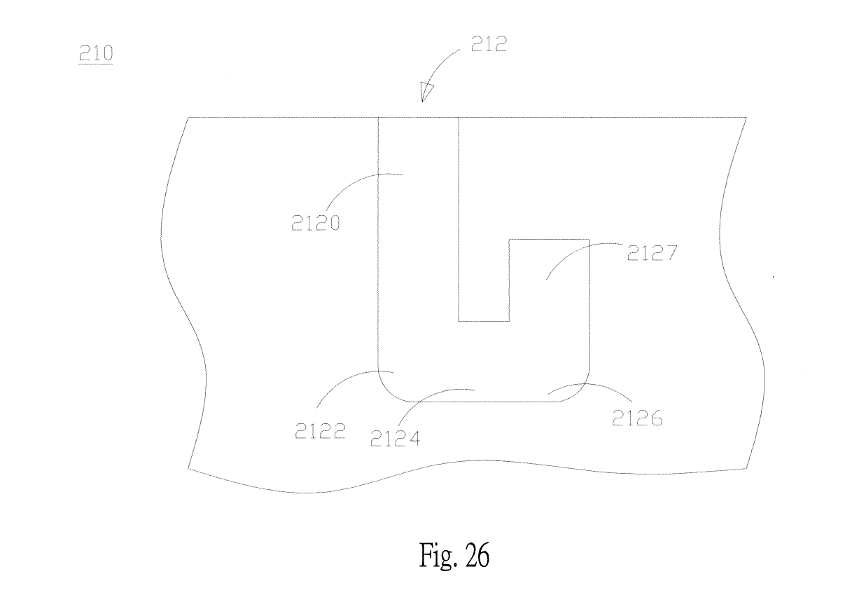

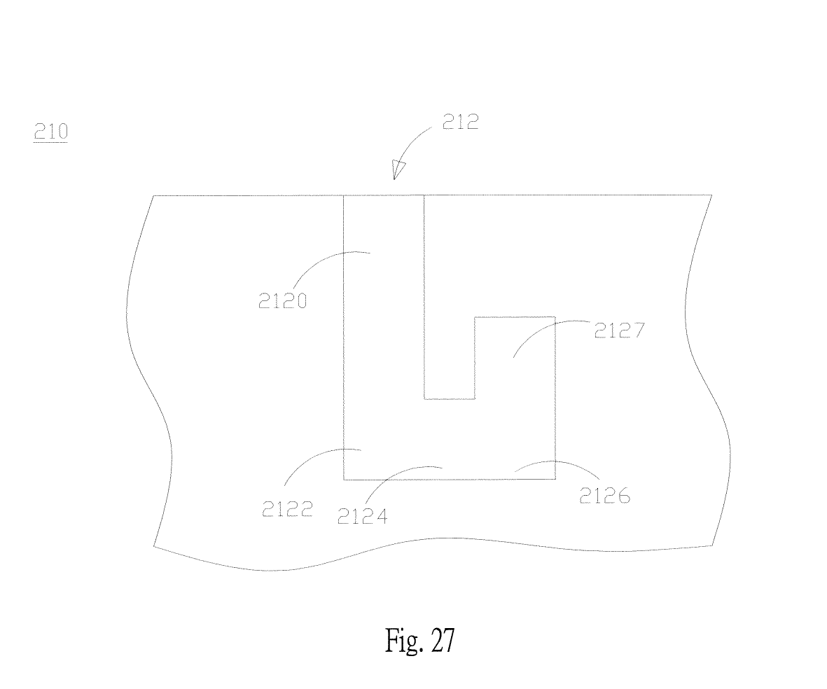

Refer to FIG. 26 and FIG. 27, a fixing groove 212 of two further embodiments (the third and the fourth) according to the present invention is revealed. As shown in the figure, the difference between the fixing groove 212 of these two embodiments and the fixing groove 212 of the above two embodiments is in that the holding and locking part 2127 of the fixing groove 212 is perpendicular to the sliding part 2124 of the fixing groove 212. The difference between the fixing groove 212 of the third embodiment in FIG. 26 and the fixing groove 212 of the fourth embodiment in FIG. 27 is in that the first turning part 2122 as well as the second turning part 2126 of the third embodiment is a fillet while the first turning part 2122 as well as the second turning part 2126 of the fourth embodiment is a right angle.

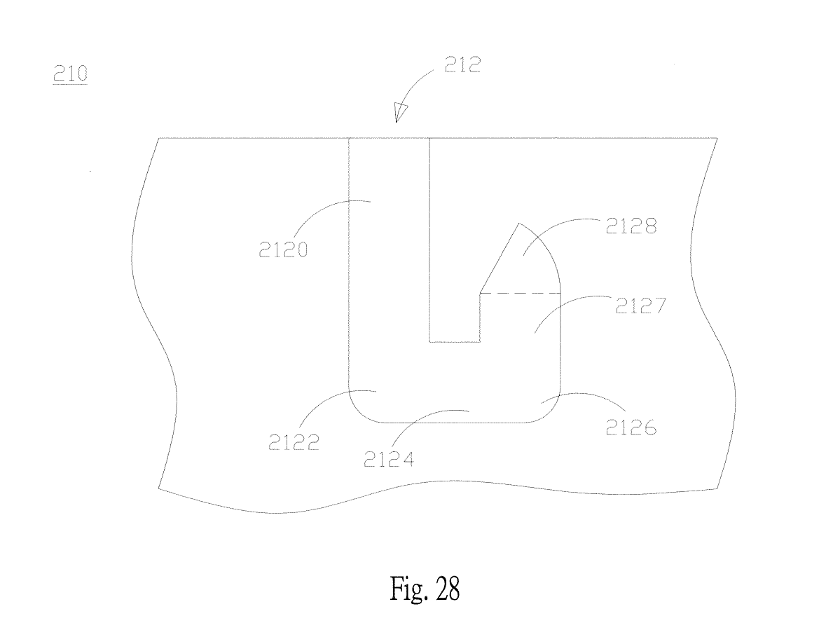

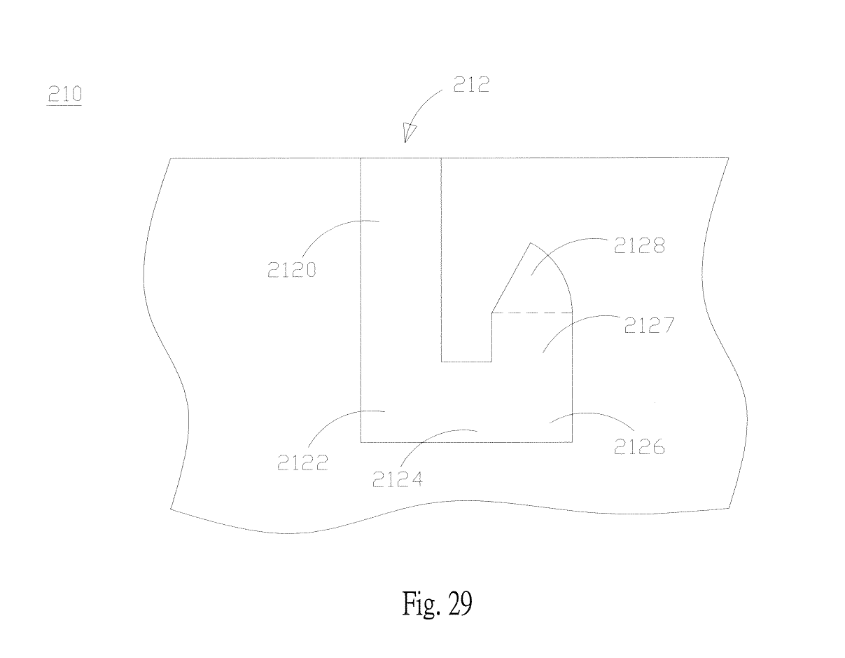

Refer to FIG. 28 and FIG. 29, schematic drawings showing a fixing groove 212 of a fifth embodiment and a fixing groove 212 of a sixth embodiment according to the present invention are revealed. The fixing groove 212 of both embodiments further includes a stopping part 2128 connected to the holding and locking part 2127. The area of the stopping part 2128 is smaller than that of the holding and locking part 2127. Thus the stopper 114 is fixed in the stopping part 2128 more tightly while the stopper 114 being turned into the holding and locking part 2127 and further slid in the stopping part 2128 due to smaller area of the stopping part 2128 than the holding and locking part 2127. Therefore higher waterproof effect is provided. Moreover, the difference between the fixing groove 212 of the fifth embodiment and the fixing groove 212 of the sixth embodiment is in that the first turning part 2122 as well as the second turning part 2126 of the fifth embodiment is a fillet while the first turning part 2122 as well as the second turning part 2126 of the sixth embodiment is a right angle.

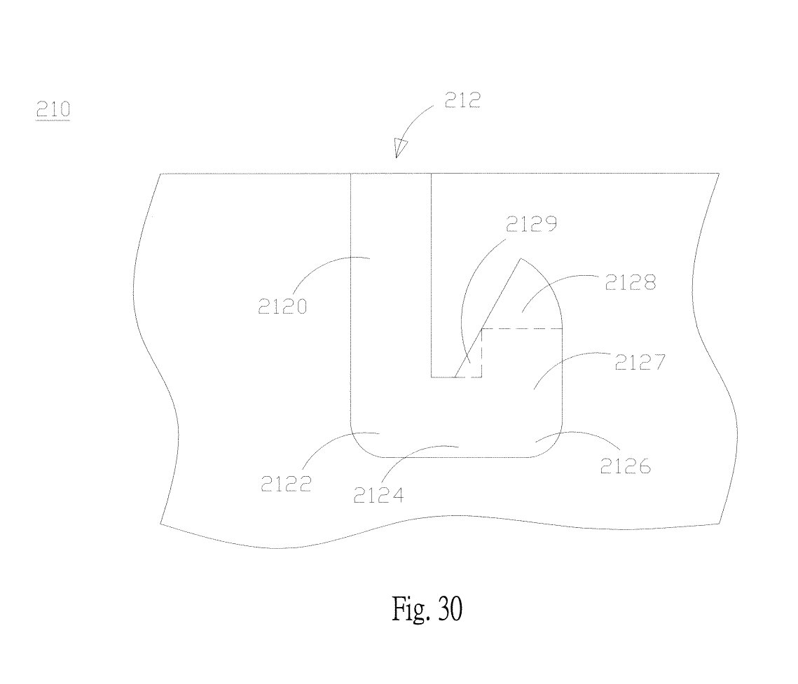

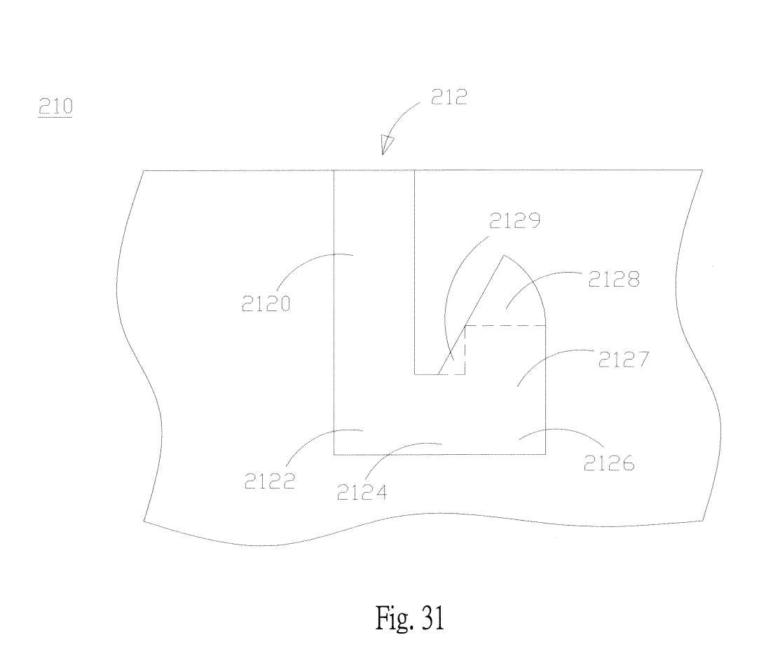

Refer to FIG. 30 and FIG. 31, schematic drawings showing a fixing groove 212 of a seventh embodiment and a fixing groove 212 of a eighth embodiment according to the present invention are revealed. As shown in the figure, the difference between the fixing groove 212 of these two embodiments and the fixing groove 212 of the above two embodiments is in that the fixing groove 212 further includes an reinforcement part 2129. The reinforcement part 2129 covers one side of the holding and locking part 2127 and one side of the stopping part 2128 adjacent to the guiding part 2120. Thereby the stopper 114 can be easily moved from the first turning part 2122 to the stopping part 2128 by a little force. The user can fix the waterproof sleeve 200 on the fixing portion 112 of the laryngoscope body 100 easily and the laryngoscope is more convenient for the user to use. Moreover, the difference between the fixing groove 212 of the seventh embodiment and the fixing groove 212 of the eighth embodiment is in that the first turning part 2122 as well as the second turning part 2126 of the seventh embodiment is a fillet while the first turning part 2122 as well as the second turning part 2126 of the eighth embodiment is a right angle.

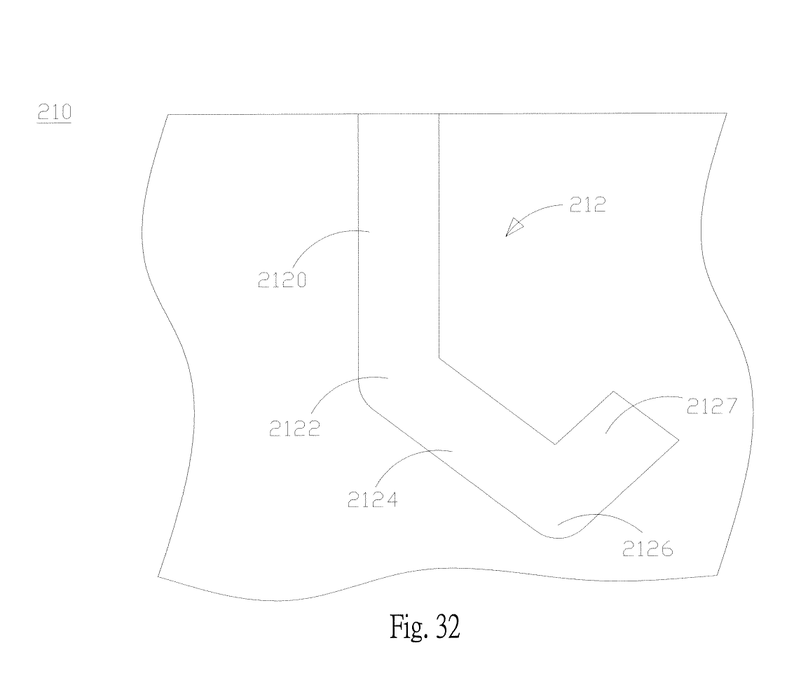

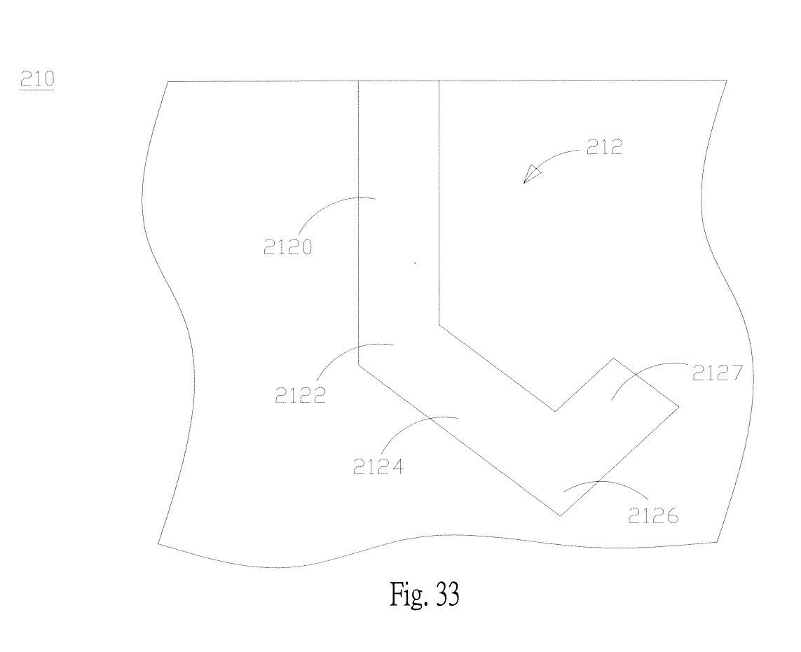

Refer to FIG. 32 and FIG. 33, schematic drawings showing a fixing groove 212 of a ninth embodiment and a fixing groove 212 of a tenth embodiment according to the present invention are revealed. As shown in the figure, the difference between the fixing groove 212 of these two embodiments and the fixing groove 212 of the above embodiments is in that a turning angle of the first turning part 2122 of the fixing groove 212 is larger than 90 degrees while a turning angle of the second turning part 2126 is 90 degrees. By the turning angle of the first turning part 2122 larger than 90 degrees, the locking strength between the stopper 114 and the fixing groove 212 is increased. Thus the handle 110 and the waterproof sleeve 200 are attached to each other more tightly. Therefore the water resistance of the laryngoscope is increased. Moreover, the difference between the fixing groove 212 of the ninth embodiment and the fixing groove 212 of the tenth embodiment is in that the first turning part 2122 as well as the second turning part 2126 of the ninth embodiment is a fillet while the first turning part 2122 as well as the second turning part 2126 of the tenth embodiment is a right angle.

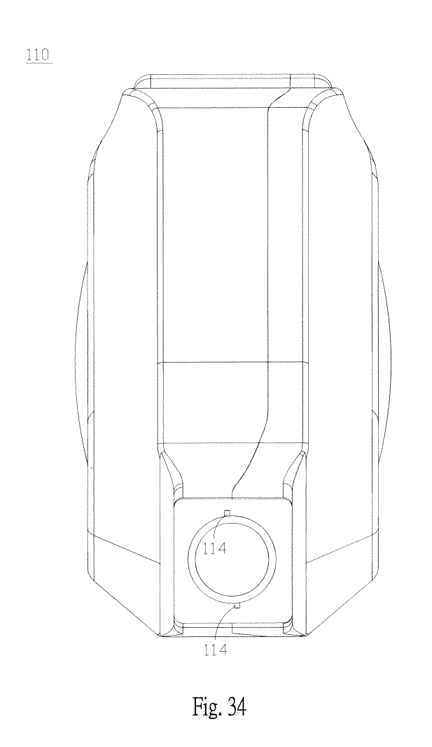

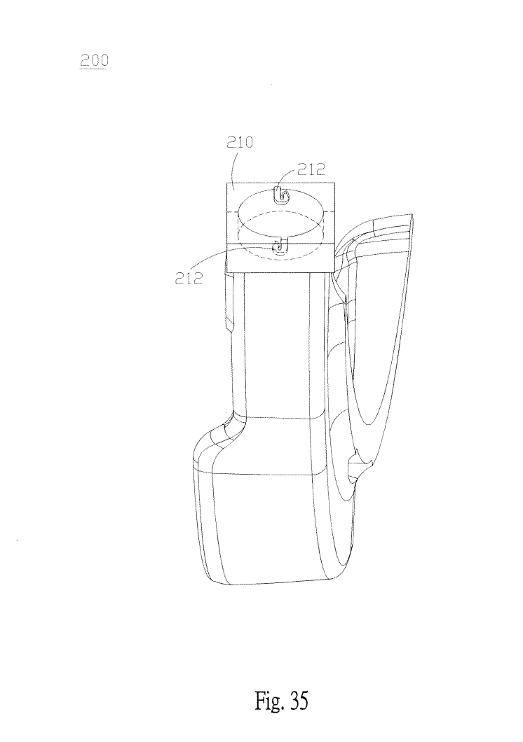

Refer to FIG. 34 and FIG. 35, a bottom view of a handle 110 and a rear view of a waterproof sleeve 200 of a further embodiment are disclosed. The difference between this embodiment and the one shown in FIG. 3 and FIG. 4 is in that the fixing portion 112 of the handle 110 is a second hollow cylinder while the connection portion 210 of the waterproof sleeve 200 is a second receiving slot. The second hollow cylinder is mounted into the second receiving slot. The stopper 114 of the fixing portion 112 is arranged at an outer sidewall of the second hollow cylinder while the fixing groove 212 is disposed on an inner sidewall of the second receiving slot. Moreover, the fixing groove 212 of this embodiment is penetrating the inner sidewall of the second receiving slot. The difference between the fixing groove 212 of this embodiment and the fixing groove 212 of the embodiment in FIG. 4 is in that the guiding part 2120 of the fixing groove 212 is disposed on the inner sidewall of the second receiving slot and crossed over the top edge of the inner sidewall thereof for guiding the stopper 114 entering into the fixing slot 212.

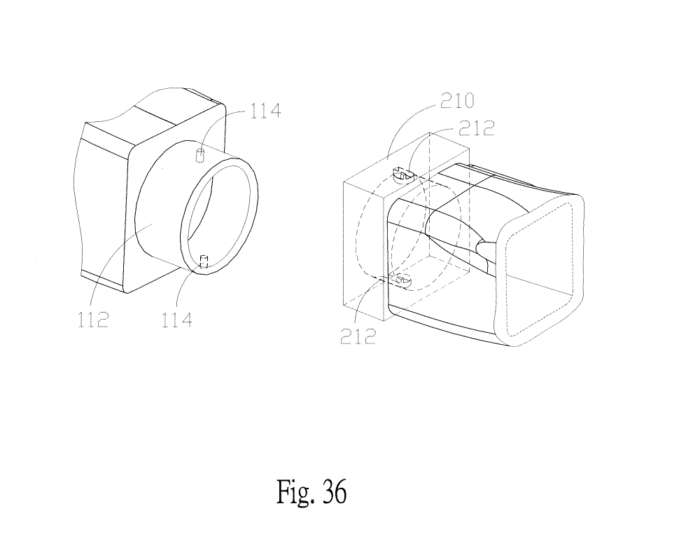

Refer to FIG. 36, an embodiment of the assembly of the handle in FIG. 34 with the waterproof sleeve in FIG. 35 is revealed. The assembly way is almost the same as that of the embodiment in FIG. 7 to FIG. 17. The only difference is in that the fixing portion 112 of the handle 110 in this embodiment is mounted into the connection portion 210 of the waterproof sleeve 200. In the embodiment shown in FIG. 4, a diameter of the fixing portion 112 of the handle 110 is large than a diameter of the connection portion 210 of the waterproof sleeve 200. In this embodiment, the diameter of the fixing portion 112 of the handle 110 is smaller than the diameter of the connection portion 210 of the waterproof sleeve 200.

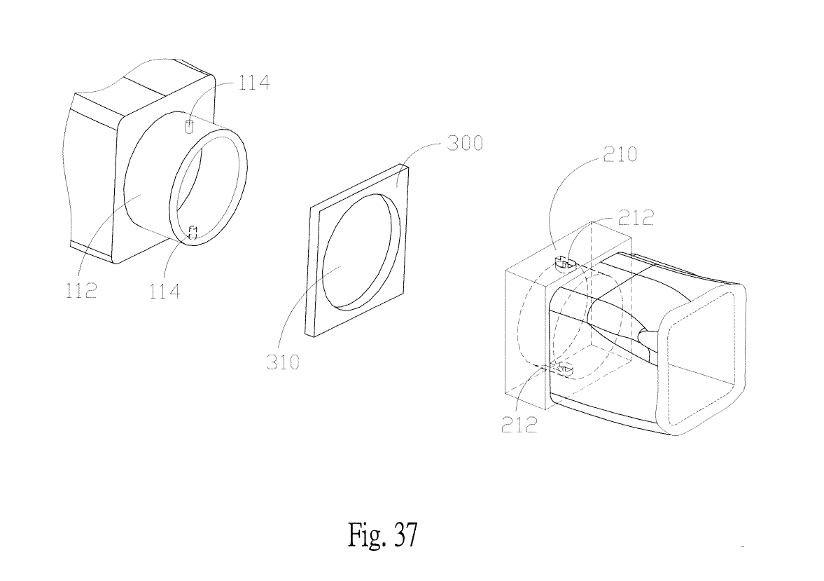

Also refer to FIG. 37, another embodiment of the assembly of the handle in FIG. 34 with the waterproof sleeve in FIG. 35 is revealed. As shown in the figure, the difference between this embodiment and the embodiment in FIG. 36 is in that the laryngoscope of this embodiment further includes a washer 300. The washer 300 is first set around the fixing portion 112 of the handle 110. Then the connection portion 210 of the waterproof sleeve 200 is sleeved over the fixing portion 112 of the handle 110 to allow the stopper 114 of the fixing portion 112 being slid inside the fixing groove 212 and locked on the rear end of the fixing groove 212.

In summary, a waterproof laryngoscope of the present invention includes a laryngoscope body and a waterproof sleeve. The laryngoscope body consists of a handle and a camera tube. The handle has a fixing portion disposed with at least one stopper while the camera tube is fixed in the handle and is extended out of the fixing portion. The waterproof sleeve is fitted over the camera tube and fixed on the handle. The waterproof sleeve includes a connection portion that is fitted over the camera tube and fixed on the fixing portion. The connection portion is arranged with at least one fixing groove. The stopper is slid in the fixing groove and locked on a rear end of the fixing groove. Thereby the patient's body fluid will not flow into the laryngoscope. The design of the laryngoscope also protects patients from being affected by toxic chemical substances and cross infection.

Thus the present invention is new, involving an inventive step and capable of industrial applications.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A waterproof laryngoscope comprising:
a laryngoscope body having
a handle arranged with a fixing portion while the fixing portion is disposed with at least one stopper; and
a camera tube fixed in the handle and extended out of the fixing portion; and
a waterproof sleeve fitted over the camera tube, fixed on the handle, and having a connection portion; the connection portion is fitted over the camera tube and fixed on the fixing portion; at least one fixing groove is disposed on the connection and the stopper is slid in the fixing groove and locked on a rear end of the fixing groove.

2. The device as claimed in claim 1, wherein the waterproof laryngoscope further includes a washer disposed with a fixing hole and the camera tube is inserted through the washer; the washer is clipped between the fixing portion and the connection portion.

3. The device as claimed in claim 1, wherein the fixing portion is a first receiving slot while the connection portion is a first hollow cylinder; the first hollow cylinder is mounted in the first receiving slot; the stopper is disposed on an inner sidewall of the first receiving slot while the fixing groove is formed on an outer sidewall of the first hollow cylinder.

4. The device as claimed in claim 3, wherein the fixing groove is not penetrating the outer sidewall of the first hollow cylinder.

5. The device as claimed in claim 3, wherein the fixing groove includes:
a guiding part disposed on the outer sidewall and crossed over a top edge of the outer sidewall;
a first turning part connected to the guiding part;
a sliding part connected to the first turning part; an angle between the sliding part and the guiding part is no less than 90 degrees;
a second turning part that is connected to the sliding part while a turning direction of the second turning part is the same as a turning direction of the first turning part; and
a holding and locking part connected to the second turning part.

6. The device as claimed in claim 5, wherein the fixing groove further includes a stopping part; the stopping part is connected to the holding and locking part while an area of the stopping part is smaller than an area of the holding and locking part.

7. The device as claimed in claim 6, wherein the fixing groove further includes a reinforcement part; the reinforcement part is connected to the holding and locking part, close to one side of the guiding part and adjacent to the stopping part.

8. The device as claimed in claim 5, wherein the first turning part as well as the second turning part is a right angle.

9. The device as claimed in claim 5, wherein the first turning part as well as the second turning part is a fillet.

10. The device as claimed in claim 1, wherein the fixing portion is a second hollow cylinder while the connection portion of the waterproof sleeve is a second receiving slot; the second hollow cylinder is mounted into the second receiving slot; the stopper is arranged at an outer sidewall of the second hollow cylinder while the fixing groove is disposed on an inner sidewall of the second receiving slot.

11. The device as claimed in claim 10, wherein the fixing groove is not penetrating the inner sidewall of the second hollow cylinder.

12. The device as claimed in claim 10, wherein the fixing groove includes:
a guiding part disposed on the inner sidewall and crossed over a top edge of the inner sidewall;
a first turning part that is connected to the guiding part;
a sliding part connected to the first turning part; an angle between the sliding part and the guiding part is no less than 90 degrees;
a second turning part that is connected to the sliding part while a turning direction of the second turning part is the same as a turning direction of the first turning part; and
a holding and locking part connected to the second turning part.

13. The device as claimed in claim 12, wherein the fixing groove further includes a stopping part; the stopping part is connected to the holding and locking part while an area of the stopping part is smaller than an area of the holding and locking part.

14. The device as claimed in claim 13, wherein the fixing groove further includes a reinforcement part; the reinforcement part is connected to the holding and locking part, close to one side of the guiding part and adjacent to the stopping part.

15. The device as claimed in claim 12, wherein the first turning part as well as the second turning part is a right angle.

16. The device as claimed in claim 12, wherein the first turning part as well as the second turning part is a fillet.

17. The device as claimed in claim 1, wherein the waterproof laryngoscope includes a plurality of stoppers arranged circularly at the fixing portion.

18. A waterproof structure comprising:
a handle having a fixing portion; the fixing portion is disposed with at least one stopper; and
a waterproof sleeve fixed on the handle and having a connection portion; the connection portion is fixed on the fixing portion and is arranged with a fixing groove; the stopper is slid in the fixing groove and is locked on a rear end of the fixing groove.

19. The structure as claimed in claim 18, wherein the waterproof structure further includes a washer disposed with a fixing hole and the connection portion is inserted through the washer; the washer is clipped between the handle and the waterproof sleeve.

20. The structure as claimed in claim 18, wherein the fixing portion is a first receiving slot while the connection portion is a first hollow cylinder; the first hollow cylinder is mounted in the first receiving slot; the stopper is disposed on an inner sidewall of the first receiving slot while the fixing groove is formed on an outer sidewall of the first hollow cylinder.

21. The structure as claimed in claim 20, wherein the fixing groove includes:
a guiding part disposed on the outer sidewall and crossed over a top edge of the outer sidewall;
a first turning part connected to the guiding part;
a sliding part connected to the first turning part; an angle between the sliding part and the guiding part is no less than 90 degrees;
a second turning part that is connected to the sliding part while a turning direction of the second turning part is the same as a turning direction of the first turning part; and
a holding and locking part connected to the second turning part.

22. The structure as claimed in claim 21, wherein the fixing groove further includes a stopping part; the stopping part is connected to the holding and locking part while an area of the stopping part is smaller than an area of the holding and locking part.

23. The structure as claimed in claim 22, wherein the fixing groove further includes a reinforcement part; the reinforcement part is connected to the holding and locking part, close to one side of the guiding part and adjacent to the stopping part.
